(19) United States
(12) Patent Application Publication
(10) Pub. No.: US 2018/0042622 A1
(43) Pub. Date: Feb. 15, 2018

(54) Anatomical Locking Guide Steel Plate, Anatomical Locking Guide Apparatus and Matched Guide Sleeve Apparatus

(71) Applicants: Peifu TANG, Beijing (CN); Lihai ZHANG, Beijing (CN); Zhe ZHAO, Beijing (CN); Xiuyun SU, Beijing (CN); Licheng ZHANG, Beijing (CN); Zhirui LI, Beijing (CN)

(72) Inventors: Peifu TANG, Beijing (CN); Lihai ZHANG, Beijing (CN); Zhe ZHAO, Beijing (CN); Xiuyun SU, Beijing (CN); Licheng ZHANG, Beijing (CN); Zhirui LI, Beijing (CN)

(21) Appl. No.: 15/663,533

(22) Filed: Jul. 28, 2017

(30) Foreign Application Priority Data
Aug. 10, 2016 (CN) 201610648185.6

(51) Int. Cl. A61B 17/17; A61B 17/80

(52) U.S. Cl. CPC A61B 17/1746; A61B 17/1728; A61B 17/8052; A61B 17/88

(57) ABSTRACT

The present disclosure relates to an anatomical locking guide steel plate for a complicated acetabulum fracture, an anatomical locking guide apparatus and matched guide sleeve apparatus. The inner side of said anatomical locking guide steel plate contacts with a face of the acetabulum, said anatomical locking guide steel plate includes three portions, a head of the steel plate, a body of the steel plate and a tail of the steel plate, said head of the steel plate, said body of the steel plate and said tail of the steel plate are jointed seamlessly. Using the steel plate provided by the present disclosure can reposition and fix the complicated acetabulum fracture efficiently. It can avoid shaping the steel plate in the surgery, so the operative time may be shortened, and the difficulty of the surgery may be lower. The surgical trauma is small, and the surgical bleeding is slight.

This application claims priority of Chinese Application No. 201610648185.6 filed on Aug. 10, 2016 and entitled “Anatomical locking guiding steel plate for complex acetabular fracture”.

### TECHNICAL FIELD

The disclosure relates to the technical field of the medical appliance, specially, it relates to an anatomical locking guide steel plate for a complicated acetabulum fracture, an anatomical locking guide apparatus for the complicated acetabulum fracture and a matched guide sleeve apparatus.

### BACKGROUND

The acetabulum fracture, particularly the Letournel-Judet type complicated acetabulum fracture which accounts 21.88% of all the acetabulum fracture, is a severe fracture trauma. The acetabulum fracture always accompanies with the fracture of other region and the trauma of the viscera, so its mortality rate and deformity rate are high, and the position of the trauma is deep resulting in the surgery is difficult. Because it involves the structure of the acetabulum anterior column and the acetabulum posterior column, the reposition and fixation are very difficult and always require jointly access from the front and the rear and fixation the front structure and the rear structure of the acetabulum at the same time. Now, there are a few methods for the reposition and fixation of the acetabulum fracture as follow.

1. Jointly access from the front and the rear and fixation using a reconstruction steel plate for the acetabulum fracture

For the complicated acetabulum fracture involving the acetabulum anterior column structure and the acetabulum posterior column structure, this method was always adopted for the fixation in the past. The surgery requires an ilium groin access from the front and a Kocher-Langenbeck access from the rear, or it requires a flared ilium femur access for revealing the front structure and the rear structure of the acetabulum at the same time. Then, a reconstruction steel plate is bent according to the anatomical form at the front edge of the minor pelvis and fixed, and a reconstruction steel plate is bent according to the anatomical form at the rear of the acetabulum posterior wall and the acetabulum posterior column and fixed.

Using this method for the fixation of the acetabulum fracture needs to reveal the front structure and the rear structure of the acetabulum at the same time, so the secondary trauma caused by the surgery is large, the quantity of the surgical bleeding is high, and the postoperative restoration is slow. Furthermore, because the ilium groin access from the front may pass through many important structural regions such as the femoral artery, the femoral vein, the femoral nerve and the like, the probability of damaging the blood vessel and the nerve is large, and the surgical danger is high. During the surgery, it needs to bend and shape two or three steel plates according to the shape of the bone. So for the surgeon without many skill, the operative time may be long, which may increase the surgical bleeding. And manually shaping the steel plate may induce large error, which is apt to cause a fracture displacement in result of the medical treatment, and the effect of the surgery is affected.

2. Access from the rear and fixation using the reconstruction steel plate combined with an anterograde anterior column pull screw for the acetabulum fracture

For the complicated acetabulum fracture involving the acetabulum anterior column structure and the acetabulum posterior column structure, a method of the Kocher-Langenbeck access from rear also can be adopted, which method can use the rear reconstruction steel plate to fix the fracture of the posterior column and use the anterograde anterior column pull screw to fix the acetabulum anterior column structure at the same time.

Compared with foresaid method of “jointly access from the front and the rear and fixation using a reconstruction steel plate for the acetabulum fracture”, this method can reduce the surgical trauma of a patient, but it also has many defects as follow. It also needs to bend and shape one or two steel plates according to the shape of the bone during the surgery, so for the surgeon without many skills, the operative time may be long, which may increase the surgical bleeding. And manually shaping the steel plate may induce large error, which is apt to cause the fracture displacement in result of the medical treatment, and the effect of the surgery is affected. During insertion of the anterograde anterior column pull screw, few anatomical marking can be referred, so when inserting the screw, many important structures such as the femoral artery, the femoral vein, the femoral nerve and the like may be damaged. Furthermore, inserting the anterograde anterior column pull screw needs to change the fluoroscopy posture repeatedly, so the radial doctor is required to be skilled, and during the surgery, the radial damage to the patient and the doctor may be great.

3. Access from the rear of the acetabulum and fixation using an anatomical locking steel plate combined with the anterograde anterior column pull screw

The acetabulum anterior column structure can be fixed by an inner fixation steel plate for the acetabulum posterior wall fracture (China Patent: CN201389072Y) and the anterograde anterior column pull screw.

By using the inner fixation steel plate for the acetabulum posterior wall fracture, it can avoid bending the steel plate in the surgery, so the operative time may be shortened, and the difficulty of the surgery may be lower. But because inserting the anterograde anterior column pull screw is carried out under the fluoroscopy, so the method also has many defects as follow. During insertion of the anterograde anterior column pull screw, few anatomical marking can be referred, so when inserting the screw, many important structures such as the femoral artery, the femoral vein, the femoral nerve and the like may be damaged. Furthermore, inserting the anterograde anterior column pull screw needs to change the fluoroscopy posture repeatedly, so the radial doctor is required to be skilled, and during the surgery, the radial damage to the patient and the doctor may be great.

4. Technique of the reconstruction steel plate at the front of the acetabulum combined with the anterograde posterior column pull screw

For many patients of the complicated acetabulum fracture, when the rear structure doesn't displace obviously and the posterior wall structure doesn't fracture, the ilium groin access can be adopted, and the reconstruction steel plate can be provided at the edge of the small pelvis. At the same time, the surgeon can insert the anterograde posterior column pull screw under the fluoroscopy.

Using this technique, when the front reconstruction steel plate is used, the ilium groin access is required, so it can't avoid to pass through the regions of the femoral artery, the vein and the nerve, and it is apt to cause an additional trauma. Furthermore, like foresaid methods, inserting the anterograde anterior column screw needs to change the fluoroscopy posture repeatedly, so the radial doctor is required to be skilled, and during the surgery, the radial damage to the patient and the doctor may be great.

To sum up, the traditional reposition and fixation operation has many defects, such as large surgical trauma, lots of bleeding, many implants, long bed ridden time of the patient, many postoperative complications and the like, so the reposition of the acetabulum fracture is still a challenge of the trauma orthopaedics.

### SUMMARY

In order to overcome the defect of the existing reposition and fixation operation of the acetabulum fracture, such as large surgical trauma, lots of bleeding, many implants, long bed ridden time of the patient and many postoperative complications, according the present disclosure, there is provided an anatomical locking guide steel plate for the complicated acetabulum fracture.

According to an aspect of the present disclosure, there is provided an anatomical locking guide steel plate for the complicated acetabulum fracture, wherein an inner side of said anatomical locking guide steel plate contacts with a face of the acetabulum, said anatomical locking guide steel plate includes three portions: a head of the steel plate, a body of the steel plate and a tail of the steel plate, said head of the steel plate, said body of the steel plate and said tail of the steel plate are jointed seamlessly,

said head of the steel plate has a triangular shape, an outer edge of said head of the steel plate is matched with the top region of the acetabulum, the inner edge of said head of the steel plate covers the ilium gluteus tuberosity and the rear region of the gluteus tuberosity,

an outer edge of said body of the steel plate is parallel to a bony edge of the acetabulum,

a tail of the steel plate tilt upwards, and its structure is matched with the structure of the hucklebone condyle,

at least one acetabulum anterior column pull screw hole, at least one Magic pull screw hole and at least one locking screw hole are provided in said head of the steel plate,

at least two quadrate zone reposition pull screw holes are provided in said body of the steel plate, and they are a first quadrate zone reposition pull screw hole and a second quadrate zone reposition pull screw hole respectively,

at least one locking screw hole is provided in said tail of the steel plate.

According to an embodiment of the present disclosure, said body of the steel plate has a width of 11 mm-17 mm.

According to another embodiment of the present disclosure, the distance from said head of the steel plate to an apex of the acetabulum is between 28 mm and 32 mm.

According to another embodiment of the present disclosure, the outer edge of said body of the steel plate is parallel to a bony edge of the acetabulum, and the distance to the bony edge of the acetabulum is 4 mm-6 mm.

According to another embodiment of the present disclosure, an angle between the central axis of said acetabulum anterior column pull screw hole and the long axis of a body is 44.6°, and the direction of the angle is downward, an angle between the central axis of said acetabulum anterior column pull screw hole and the coronary axis of the body is 56.7°, and the direction of the angle is inward, an angle between the central axis of said acetabulum anterior column pull screw hole and the sagittal axis of the body is 64.1°, and the direction of the angle is forward.

According to another embodiment of the present disclosure, an angle between the central axis of said Magic pull screw hole and the long axis of the body is 49.1°, and the direction of the angle is downward, an angle between the central axis of said Magic pull screw hole and the coronary axis of the body is 55.6°, and the direction of the angle is inward, an angle between the central axis of said Magic pull screw hole and the sagittal axis of the body is 59.9°, and the direction of the angle is backward.

According to another embodiment of the present disclosure, said first quadrate zone reposition pull screw hole is located at the ilium side, and said second quadrate zone reposition pull screw hole is located at the ischium side;

An angle between the central axis of said first quadrate zone reposition pull screw hole and the long axis of the body is 88.6°, and the direction of the angle is downward, an angle between the central axis of said first quadrate zone reposition pull screw hole and the coronary axis of the body is 32.0°, and the direction of the angle is inward, an angle between the central axis of said first quadrate zone reposition pull screw hole and the sagittal axis of the body is 58.0°, and the direction of the angle is forward, an angle between the central axis of said first quadrate zone reposition pull screw hole and the plane of the quadrate zone is 130°;

an angle between the central axis of said second quadrate zone reposition pull screw hole and the long axis of the body is 87.4°, and the direction of the angle is downward, an angle between the central axis of said second quadrate zone reposition pull screw hole and the coronary axis of the body is 24.38°, and the direction of the angle is inward, an angle between the central axis of said second quadrate zone reposition pull screw hole and the sagittal axis of the body is 65.8°, and the direction of the angle is forward, an angle between the central axis of said second quadrate zone reposition pull screw hole and the plane of the quadrate zone is 123°.

According to another embodiment of the present disclosure, a first locking screw hole is provided in said head of the steel plate, and a third locking screw hole is provided in said tail of the steel plate;

an angle between the central axis of said first locking screw hole and the long axis of the body is 72.3°, and the direction of the angle is downward, an angle between the central axis of said first locking screw hole and the coronary axis of the body is 66.8°, and the direction of the angle is inward, an angle between the central axis of said first locking screw hole and the sagittal axis of the body is 27.9°, and the direction of the angle is forward;

an angle between the central axis of said third locking screw hole and the long axis of the body is 57.8°, and the direction of the angle is downward, an angle between the central axis of said third locking screw hole and the coronary axis of the body is 48.8°, and the direction of the angle is inward, an angle between the central axis of said third locking screw hole and the sagittal axis of the body is 58.0°, and the direction of the angle is forward.

According to another embodiment of the present disclosure, a second locking screw hole is provided in said head of the steel plate, and said second locking screw hole is located under said first locking screw hole;

a fourth locking screw hole is provided in the tail of the steel plate, and said fourth locking screw hole is located under said third locking screw hole;

an angle between the central axis of said second locking screw hole and the long axis of the body is 88.0°, and the direction of the angle is downward, an angle between the central axis of said second locking screw hole and the coronary axis of the body is 48.3°, and the direction of the angle is inward, an angle between the central axis of said second locking screw hole and the sagittal axis of the body is 41.8°, and the direction of the angle is forward,

the central axis of said fourth locking screw hole is parallel to the central axis of said third locking screw hole.

According to another aspect of the present disclosure, an anatomical locking guide apparatus for a complicated acetabulum fracture is provided. Said apparatus includes the anatomical locking guide steel plate for the complicated acetabulum fracture according to any one of the claims 1-9 and a guide module,

a first connection hole is provided between the first quadrate zone reposition pull screw hole and the second quadrate zone reposition pull screw hole,

a groove is provided in the ventral side of said guide module for mating with said body of the steel plate, so as to combine with said steel plate,

a first guide screw hole, a second guide screw hole and a second connection hole are provided in said guide module, the first guide screw hole, the second guide screw hole and the second connection hole correspond to the first quadrate zone reposition pull screw hole, the second quadrate zone reposition pull screw hole and the first connection hole respectively.

an inner edge of said guide module is matched with the big incisura of the ischium.

According to another aspect of the present disclosure, a guide sleeve apparatus is provided, said guide sleeve apparatus can suitably use with aforesaid anatomical locking guide steel plate for the complicated acetabulum fracture or aforesaid anatomical locking guide apparatus for the complicated acetabulum fracture,

said guide sleeve apparatus includes an outer sleeve, a pointed cone puncturing element and an inner core,

an inner diameter of said outer sleeve is matched with the outer diameters of said pointed cone puncturing element and said inner core, thread is provided at the front end of said outer sleeve, and the thread is matched with the screw holes in said anatomical locking guide steel plate and/or said guide module,

the head of said pointed cone puncturing element is a blunt nosed cone,

said inner core has a hollow structure, it's inner diameter is matched with the outer diameter of a guide needle, the head of said inner core has a serrate structure with a slope.

The anatomical locking guide steel plate for the complicated acetabulum fracture provided by the present disclosure is a preformed steel plate, it can tightly cling to the surface of the fracture site at the rear of the acetabulum, so it can offer an inner fixation stably and reliably. Using the anatomical locking guide steel plate can obviate the defect in the prior art and resolve the problem of longer surgical time and larger shaping error in result of shaping in the surgery. The anatomical locking guide steel plate provided by the present disclosure can tightly cling to the bone, so the bone configuration at the rear of the acetabulum can be used as a positioning feature, and it can offer a precise insertion position for anterogradely inserting the acetabulum anterior column pull screw. During the actual surgery, after the anatomical locking guide steel plate clings to the acetabulum, it is enough that various screws are inserted according to the screw holes in the anatomical locking guide steel plate. The insertion position is accurate, and other tissue can't be damaged. It doesn't require repeated fluoroscopy, so the amount of radiation received by the patient and the doctor can be reduced effectively.

### BRIEF DESCRIPTION OF THE FIGURES

Other features, objects and advantages of the present disclosure will become more apparent by reading the detailed description of non-restrictive embodiments taken in conjunction with the accompanying figures, wherein:

FIG. 1 is a structural schematic view showing a specific embodiment of an anatomical locking guide steel plate for a complicated acetabulum fracture according to the present disclosure.

FIG. 2 is a structural schematic view showing a specific embodiment of a guide module according to the present disclosure.

FIG. 3 is a structural schematic view showing a specific embodiment of the anatomical locking guide apparatus for the complicated acetabulum fracture according to the present disclosure.

FIG. 4 is a structural schematic view showing another specific embodiment of the anatomical locking guide apparatus for the complicated acetabulum fracture according to the present disclosure.

FIG. 5 is a structural schematic view showing a specific embodiment of a guide sleeve apparatus according to the present disclosure.

In the figures, same or like reference characters are used to represent same or like parts.

### DETAILED DESCRIPTION

Hereinafter, many different embodiments or examples will be described for embodying different structure of the present disclosure. For the purpose of simplifying the disclosure of the present disclosure, components and installation of specific examples are described hereinafter. Furthermore, reference numbers and/or letters may be repeated in the different embodiments of the present disclosure. The purpose of this repetition is simplification and clarity, and there is no purpose of indicating the relation of said embodiments and/or installation. It is noted that the components shown in the figures can be drawn not to scale. The well-known parts and processing technique and technology are omitted in order not to unnecessarily limit the present disclosure.

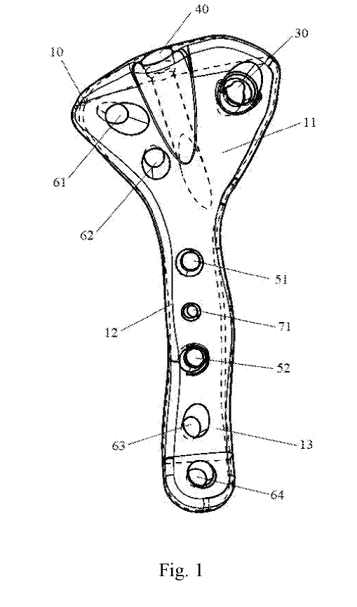

With reference to FIG. 1, FIG. 1 is a structural schematic view showing a specific embodiment of the anatomical locking guide steel plate for the complicated acetabulum fracture according to the present disclosure. An inner side of said anatomical locking guide steel plate 10 contacts with a surface of the acetabulum. Said anatomical locking guide steel plate includes three portions: a head 11 of the steel plate, a body 12 of the steel plate and a tail 13 of the steel plate, the head 11 of the steel plate, the body 12 of the steel plate and the tail 13 of the steel plate are jointed seamlessly. Preferentially, the head 11 of the steel plate, the body 12 of the steel plate and the tail 13 of the steel plate can form integrally. Furthermore, the anatomical locking guide steel plate 10 can be made of medical steel, it also can be made of other medical high molecular material which has certain hardness and can used in medical field.

Said head 11 of the steel plate has a triangular shape, an outer edge of said head 11 of the steel plate is matched with a top region of the acetabulum, an inner edge of said head 11 of the steel plate covers the ilium gluteus tuberosity and the rear region of the gluteus tuberosity. The head 11 of the steel plate can offer enough fixed force for the acetabulum, so the condition that the top of the acetabulum displaces upwards because of load is avoided. Preferentially, the distance from the head of the steel plate to an apex of the acetabulum is between 28 mm and 32 mm. According to the result from massive data analysis, it shows that for the most of the patient, the head of the steel plate can offer the strongest fixed force when the distance from the head 11 of the steel plate to the apex of the acetabulum is 30 mm. What is to say, the reposition and fixation effect of the anatomical locking guide steel plate 10 is best.

An outer edge of said body 12 of the steel plate is parallel to a bony edge of the acetabulum, and the distance to the bony edge of the acetabulum is 4 mm-6 mm. Preferentially, the distance from the outer edge of the body 12 of the steel plate to the bony edge of the acetabulum is 5 mm. Said body of the steel plate has a width of 11 mm-17 mm. According to the analysis of massive samples of the complicated acetabulum fracture by the inventor, preferentially, the width of the body 12 of the steel plate is 14 mm.

A tail 13 of the steel plate tilt upwards, and its structure is matched with the structure of the hucklebone condyle. As seen in FIG. 1, the whole shape of the anatomical locking guide steel plate 10 is matched with the rear structure of the acetabulum. The anatomical locking guide steel plate 10 can cling to the acetabulum seamlessly, and it can precisely reposition the acetabulum fracture.

Wherein, the shape of the head 11 of the steel plate, the shape of the body 12 of the steel plate and the shape of the tail 13 of the steel plate are determined by the inventor according to the analysis of bony data of thousands persons, so they can be matched with the acetabulum shapes of all the people essentially. Furthermore, as well known, the acetabulum is divided into a left portion and a right portion. To avoid redundancy, the steel plate of one side (right side) is illustrated as an example hereinafter. But the left side is similar to the right side essentially, With reference to the context, the ordinary skill in the art will readily know the detail of the left steel plate as the anatomical locking guide steel plate for the complicated acetabulum fracture.

At least one acetabulum anterior column pull screw hole 30, at least one Magic pull screw hole 40 and at least one locking screw hole are provided in the head 11 of the steel plate. An acetabulum anterior column pull screw is inserted anterogradely through the acetabulum anterior column pull screw hole 30, a Magic pull screw is inserted anterogradely through the Magic pull screw hole 40. Similarly, a locking screw is inserted anterogradely through the locking screw hole.

At least two quadrate zone reposition pull screw holes are provided in said body 12 of the steel plate, and they are a first quadrate zone reposition pull screw hole 51 and a second quadrate zone reposition pull screw hole 52 respectively. A first quadrate zone reposition pull screw and a second quadrate zone reposition pull screw are inserted anterogradely through the first quadrate zone reposition pull screw hole 51 and the second quadrate zone reposition pull screw hole 52, respectively.

At least one locking screw hole is provided in said tail 13 of the steel plate, and a locking screw is inserted anterogradely through the locking screw hole.

The positions of various screw holes in the disclosure are determined elaborately by the inventor, so insert points of various screws are optimal. More importantly, because different screws have different function and feature, the sections of the screw holes aren't the same each other, and the directions of the screw holes aren't perpendicular to the anatomical locking guide steel plate 10. The directions of the screw holes have certain angles to the anatomical locking guide steel plate 10, and the concrete angular values are described respectively hereinafter. Furthermore, for the purpose of illustrating the direction of each screw hole in detail, now we explain the technical terms described hereinafter. A long axis of a body is referred to a linear direction from top to bottom when a man is upstanding, a coronary axis of the body is referred to a linear direction from left to right when a main is upstanding, and a sagittal axis of the body is referred to a linear direction from front to rear.

An angle between the central axis of said acetabulum anterior column pull screw hole 30 and the long axis of the body is 44.6°, and the direction of the angle is downward. An angle between the central axis of said acetabulum anterior column pull screw hole and the coronary axis of the body is 56.7°, and the direction of the angle is inward. An angle between the central axis of said acetabulum anterior column pull screw hole and the sagittal axis of the body is 64.1°, and the direction of the angle is forward. After the acetabulum anterior column pull screw is inserted along the acetabulum anterior column pull screw hole 30, it can work to reposition the acetabulum fracture. In particular, the section of the acetabulum anterior column pull screw hole 30 has a special shape. The acetabulum anterior column pull screw hole 30 has a threaded tack hole on the surface side. Preferentially, the bottom diameter of the acetabulum anterior column pull screw hole 30 is 6.5 mm. There is a spherical surface at the deep side of the thread, and the spherical surface is matched with the tail of the acetabulum anterior column pull screw. There is a cylindrical surface at the deep side of the hole, and the acetabulum anterior column pull screw can pass through the cylindrical surface. Preferentially, the diameter of said cylindrical surface is 5.0 mm.

An angle between the central axis of said Magic pull screw hole 40 and the long axis of the body is 49.1°, and the direction of the angle is downward. An angle between the central axis of said Magic pull screw hole and the coronary axis of the body is 55.6°, and the direction of the angle is inward. An angle between the central axis of said Magic pull screw hole and the sagittal axis of the body is 59.9°, and the direction of the angle is backward. After the Magic pull screw is inserted along the Magic pull screw hole 40, it can work to reposition the acetabulum fracture. In particular, the section of the Magic pull screw hole 40 has a special shape, and it is similar to that of the acetabulum anterior column pull screw hole 30. The Magic pull screw hole 40 has a threaded tack hole on the surface side. Preferentially, the bottom diameter of the Magic pull screw hole 40 is 6.5 mm. There is a spherical surface at the deep side of the thread, and the spherical surface is matched with the tail of the Magic pull screw. There is a cylindrical surface at the deep side of the hole, and the Magic pull screw can pass through the cylindrical surface. Preferentially, the diameter of said cylindrical surface is 5.0 mm.

Said first quadrate zone reposition pull screw hole 51 is located at the ilium side. An angle between the central axis of said first quadrate zone reposition pull screw hole 51 and the long axis of the body is 88.6°, and the direction of the angle is downward. An angle between the central axis of said first quadrate zone reposition pull screw hole 51 and the coronary axis of the body is 32.0°, and the direction of the angle is inward. An angle between the central axis of said first quadrate zone reposition pull screw hole 51 and the sagittal axis of the body is 58.0°, and the direction of the angle is forward. An angle between the central axis of said first quadrate zone reposition pull screw hole 51 and the plane of the quadrate zone is 130°. And the first quadrate zone reposition pull screw is inserted through the first quadrate zone reposition pull screw hole 51.

Said second quadrate zone reposition pull screw hole 52 is located at the ischium side. An angle between the central axis of said second quadrate zone reposition pull screw hole 52 and the long axis of the body is 87.4°, and the direction of the angle is downward. An angle between the central axis of said second quadrate zone reposition pull screw hole 52 and the coronary axis of the body is 24.38°, and the direction of the angle is inward. An angle between the central axis of said second quadrate zone reposition pull screw hole 52 and the sagittal axis of the body is 65.8°, and the direction of the angle is forward. An angle between the central axis of said second quadrate zone reposition pull screw hole 52 and the plane of the quadrate zone is 123°. And the second quadrate zone reposition pull screw is inserted through the second quadrate zone reposition pull screw hole 52.

The first quadrate zone reposition pull screw and the second quadrate zone reposition pull screw are used to fix the anatomical locking guide steel plate on the acetabulum, and their main function is to reposition and fix the fracture of the quadrate zones. Preferentially, the first quadrate zone reposition pull screw and the second quadrate zone reposition pull screw are hollow pull screws which have a diameter of 4 mm. Preferentially, the sectional structures of said first quadrate zone reposition pull screw hole 51 and said second quadrate zone reposition pull screw hole 52 are the same as the sectional structure of the acetabulum anterior column pull screw hole 30. Because the diameters of the first quadrate zone reposition pull screw and the second quadrate zone reposition pull screw are smaller slightly, the sectional sizes of said first quadrate zone reposition pull screw hole 51 and said second quadrate zone reposition pull screw hole 52 are smaller than the sectional size of the acetabulum anterior column pull screw hole 30 in the same ratio.

For the purpose of fixing the anatomical locking guide steel plate 10 on the acetabulum and avoiding displacement, a first locking screw hole 61 is provided in the head of the steel plate, and a third locking screw hole 63 is provided in the tail of the steel plate.

An angle between the central axis of said first locking screw hole 61 and the long axis of the body is 72.3°, and the direction of the angle is downward. An angle between the central axis of said first locking screw hole 61 and the coronary axis of the body is 66.8°, and the direction of the angle is inward. An angle between the central axis of said first locking screw hole 61 and the sagittal axis of the body is 27.9°, and the direction of the angle is forward. The first locking screw hole 61 directs to the front and inferior crest of the ilium where the bone is hard, so the first locking screw hole 61 mainly works to stabilize the anatomical locking guide steel plate 10.

An angle between the central axis of said third locking screw hole 63 and the long axis of the body is 57.8°, and the direction of the angle is downward. An angle between the central axis of said third locking screw hole 63 and the coronary axis of the body is 48.8°, and the direction of the angle is inward. An angle between the central axis of said third locking screw hole 63 and the sagittal axis of the body is 58.0°, and the direction of the angle is forward.

Preferentially, for the purpose of further enhancing the joint stabilization of the anatomical locking guide steel plate 10 and the acetabulum, a second locking screw hole 62 is provided in the head 11 of the steel plate, and said second locking screw hole 62 is located under said first locking screw hole 61. A fourth locking screw hole 64 is provided in the tail 13 of the steel plate, and said fourth locking screw hole 64 is located under said third locking screw hole 63.

An angle between the central axis of said second locking screw hole 62 and the long axis of the body is 88.0°, and the direction of the angle is downward. An angle between the central axis of said second locking screw hole 62 and the coronary axis of the body is 48.3°, and the direction of the angle is inward. An angle between the central axis of said second locking screw hole 62 and the sagittal axis of the body is 41.8°, and the direction of the angle is forward. The second locking screw hole 62 mainly works to share the vertical pressure at the top of the acetabulum.

The central axis of said fourth locking screw hole 64 is parallel to the central axis of said third locking screw hole 63.

Preferentially, the section shapes of the first locking screw hole 61, the second locking screw hole 62, the third locking screw hole 63 and the fourth locking screw hole 64 are similar to that of the acetabulum anterior column pull screw hole 30, and their sizes are matched with that of the locking screws.

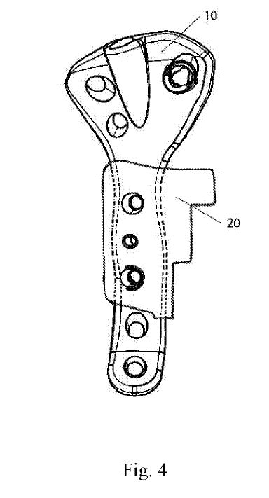

In practical application, an anatomical locking guide apparatus is also used to reposition and fix the acetabulum fracture. With reference to FIG. 3 and FIG. 4, the apparatus includes aforesaid anatomical locking guide steel plate 10 and a guide module 20 for the complicated acetabulum fracture. An inner edge of the guide module 20 is matched with the big incisura of the ischium, so by the aid of the guide module 20, the anatomical locking guide steel plate 10 can be positioned accurately and quickly, and the surgery can be perform efficiently and precisely.

For the purpose of combining with the guide module 20, a first connection hole 71 is provided between said first quadrate zone reposition pull screw hole 51 and said second quadrate zone reposition pull screw hole 52.

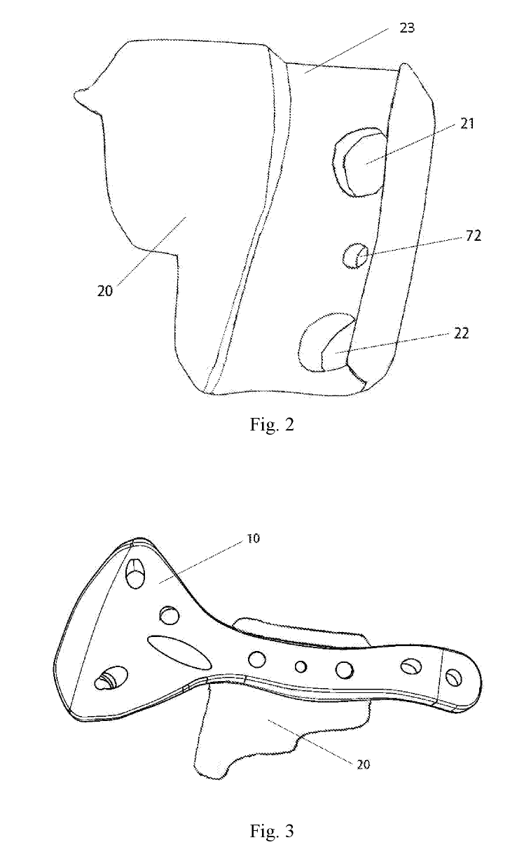

With reference to FIG. 2, FIG. 2 is a structural schematic view showing a specific embodiment of the guide module according to the present disclosure. A groove 23 is provided in the ventral side of said guide module 20 for mating with said body 12 of the steel plate, so as to combine with said anatomical locking guide steel plate 10.

A first guide screw hole 21, a second guide screw hole 22 and a second connection hole 72 are provided in the guide module 20. The first guide screw hole 21, the second guide screw hole 22 and the second connection hole 72 correspond to the first quadrate zone reposition pull screw hole 51, the second quadrate zone reposition pull screw hole 52 and the first connection hole 71 respectively. Because the angles and the directions of the first quadrate zone reposition pull screw hole 51, the second quadrate zone reposition pull screw hole 52 and the first connection hole 71 are described in detail hereinbefore, the first guide screw hole 21, the second guide screw hole 22 and the second connection hole 72 aren't described redundantly. The first connection hole 71 and the second connection hole 72 mainly work to position and connect the anatomical locking guide steel plate 10 and the guide module 20, so it is enough that they are matched each other.

Preferentially, said first guide screw hole 21 and the second guide screw hole 22 are coaxial with the first quadrate zone reposition pull screw hole 51 and the second quadrate zone reposition pull screw hole 52 respectively. Optionally, the diameters of the first guide screw hole 21 and the second guide screw hole 22 are both 8 mm. The first guide screw hole 21 and the second guide screw hole 22 both have smooth and cylindrical inner surface where it is enough that the screw can pass through, so the thread is needless. If you want to improve the effect of fixation, the thread can be added on their inner surface. If the thread is added, the inner diameter may be adjusted to rotatably connect with the screw seamlessly.

Preferentially, the diameters of the first connection hole 71 and the second connection hole 72 are both 3.5 mm, and the anatomical locking guide steel plate 10 and the guide module 20 are connected by a screw which has a thread diameter of 3.5 mm.

In order that aforesaid screws can be inserted into predetermined positions precisely and quickly, according to the present disclosure, a guide sleeve apparatus is provided to suitably use with the anatomical locking guide steel plate for the complicated acetabulum fracture or the anatomical locking guide apparatus for the complicated acetabulum fracture.

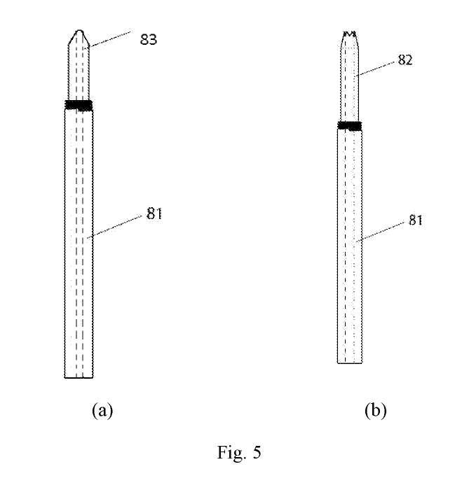

As seen in FIG. 5, said guide sleeve apparatus includes an outer sleeve 81, a pointed cone puncturing element 83 and an inner core 82. Wherein, FIG. 5(a) shows that the pointed cone puncturing element is inserted into the outer sleeve 81; FIG. 5(b) shows that the pointed cone puncturing element has been drawn out, and the inner core 82 is inserted into the outer sleeve 81.

The inner diameter of said outer sleeve 81 is matched with the outer diameters of said pointed cone puncturing element and said inner core 82. Thread is provided at the front end of said outer sleeve 81, and the thread is matched with the screw holes in said anatomical locking guide steel plate 10 and/or said guide module 20. Preferentially, the outer diameter of the outer sleeve is 7.5 mm, the inner diameter thereof is 5.1 mm, and the inner core 82 and the pointed cone puncturing element which have outer diameters of 5 mm can pass through. The outer diameter of the tail thereof is 13 mm. Thread which has a bottom diameter of 6.5 mm is provided at the front end, the thread is matched with the thread at the front end of aforesaid acetabulum anterior column pull screw hole 30, so it ensures that the direction of the outer sleeve 81 is the predetermined insertion direction of the anatomical locking guide steel plate 10.

The head of said pointed cone puncturing element is a blunt nosed cone. Preferentially, the diameter of the pointed cone puncturing element is 5 mm. after the pointed cone puncturing element is inserted into the outer sleeve 81, its front end protrudes out, so it can be used for positioning. Preferentially, a handle is provided at the terminal end of the pointed cone puncturing element, for the purpose of easy grasp.

Said inner core 82 has a hollow structure, its inner diameter is matched with the outer diameter of a guide needle. The head of said inner core 82 has a serrate structure with a slope. Preferentially, the outer diameter of said inner core 82 is 5 mm, the inner diameter thereof is 2.1 mm, and the guide needle which has a diameter of 2.0 mm can pass through. The outer diameter of the tail thereof is 13 mm. Similarly to the pointed cone puncturing element, after the inner core 82 is inserted into the outer sleeve 81 and assembled, its front portion protrudes out.

In order to further specify the use method of the guide sleeve apparatus, now an example is described. First, during the surgery, an incision which has a length of about 20 mm is made in the skin. The outer sleeve 81 and the pointed cone puncturing element are assembled, and the assembly obtusely punctures the subcutaneous tissue and the muscle and up to a screw hole (the screw hole is one of aforesaid screw holes). The outer sleeve 81 rotates to connect with the screw hole. Thereafter, the pointed cone puncturing element is drawn out, and the inner core 82 is inserted into the outer sleeve 81. At this time, the cortical bone can be drilled through by a bone drill, and a guide needle can be put in. Finally, the inner core 82 is drawn out, and a screw matched with aforesaid screw hole is put in. The screw hole described in this paragraph may be the acetabulum anterior column pull screw hole 30, the Magic pull screw hole 40, the first quadrate zone reposition pull screw hole 51, the second quadrate zone reposition pull screw hole 52, the first locking screw hole 61, the second locking screw hole 62, the third locking screw hole 63 and/or the fourth locking screw hole 64 as stated above.

The sizes of many sites in the anatomical locking guide steel plate 10, the guide module 20 and the guide sleeve apparatus are illustrated hereinbefore, but the person skilled in the art should understand that aforesaid examples aren't intended to limit the technical scheme. According to demand, the person skilled in the art can correspondingly change the sizes of the screw, the screw hole, the outer sleeve, the inner core, the pointed cone puncturing element, the guide needle and the like, so that they can meet the actual demand optimally. At the same time, it is noted that the central axis angles of various screw holes are acquired by the inventor according to the data analysis of massive samples, and it ensures that the tissue such as the artery, the nerve plexus and so on can't be damaged on the basis of the reposition and fixation of the acetabulum fracture, so these angles can't be any changed.

The anatomical locking guide steel plate provided by the present disclosure can offer the insertion position and direction for each screw itself, so the damage to the doctor and the patient because of repeated fluoroscopy in the surgery can be avoided. It can reduce the risk of damage to the nerve and the artery because of the directional error of insertion, and it can reposition and fix the complicated acetabulum fracture precisely, quickly and safely, so it is beneficial to the postoperative restoration for the patient. Furthermore, the present disclosure also provides a guide module and/or a guide sleeve apparatus combined with the anatomical locking guide steel plate. Using the auxiliary appliance can further increase the surgical efficiency and the success ratio, reduce the surgical time, and make the reposition and the fixation of the acetabulum fracture more accurate and safer.

Although the exemplary embodiments and their advantages have been described in detail, it should be understood that various changes, substitutions, and alterations can be made in these embodiments without departing from the spirit of the disclosure and the protected scope of the appended claims. With regard to the other examples, as one of ordinary skill in the art will readily appreciate, the sequence of the processing steps can be varied without departing from the protected scope of the disclosure.

Moreover, the scope of the present application is not intended to be limited to the particular embodiments of the process, machine, manufacture, composition of matter, means, methods, and steps described in the specification. As one of ordinary skill in the art will readily appreciate from the disclosure of the present disclosure, the processes, machines, manufacture, compositions of matter, means, methods, or steps presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An anatomical locking guide steel plate for a complicated acetabulum fracture, wherein, an inner side of said anatomical locking guide steel plate contacts with a surface of the acetabulum, said anatomical locking guide steel plate includes three portions: a head of the steel plate, a body of the steel plate and a tail of the steel plate, said head of the steel plate, said body of the steel plate and said tail of the steel plate are jointed seamlessly,
said head of the steel plate has a triangular shape, an outer edge of said head of the steel plate is matched with a top region of the acetabulum, an inner edge of said head of the steel plate covers the ilium gluteus tuberosity and the rear region of the gluteus tuberosity,
an outer edge of said body of the steel plate is parallel to a bony edge of the acetabulum,
a tail of the steel plate tilt upwards, and its structure is matched with the structure of the hucklebone condyle,
at least one acetabulum anterior column pull screw hole, at least one Magic pull screw hole and at least one locking screw hole are provided in said head of the steel plate,
at least two quadrate zone reposition pull screw holes are provided in said body of the steel plate, and they are a first quadrate zone reposition pull screw hole and a second quadrate zone reposition pull screw hole respectively,
at least one locking screw hole is provided in said tail of the steel plate.

2. The anatomical locking guide steel plate according to claim 1, wherein, said body of the steel plate has a width of 11 mm-17 mm.

3. The anatomical locking guide steel plate according to claim 1, wherein, the distance from said head of the steel plate to an apex of the acetabulum is 28 mm-32 mm.

4. The anatomical locking guide steel plate according to claim 1, wherein, the outer edge of said body of the steel plate is parallel to a bony edge of the acetabulum, and the distance to the bony edge of the acetabulum is 4 mm-6 mm.

5. The anatomical locking guide steel plate according to claim 1, wherein, an angle between the central axis of said acetabulum anterior column pull screw hole and the long axis of the body is 44.6°, and the direction of the angle is downward; an angle between the central axis of said acetabulum anterior column pull screw hole and the coronary axis of the body is 56.7°, and the direction of the angle is inward; an angle between the central axis of said acetabulum anterior column pull screw hole and the sagittal axis of the body is 64.1°, and the direction of the angle is forward.

6. The anatomical locking guide steel plate according to claim 1, wherein, an angle between the central axis of said Magic pull screw hole and the long axis of the body is 49.1°, and the direction of the angle is downward; an angle between the central axis of said Magic pull screw hole and the coronary axis of the body is 55.6°, and the direction of the angle is inward; an angle between the central axis of said Magic pull screw hole and the sagittal axis of the body is 59.9°, and the direction of the angle is backward.

7. The anatomical locking guide steel plate according to claim 1, wherein, said first quadrate zone reposition pull screw hole is located at the ilium side, and said second quadrate zone reposition pull screw hole is located at the ischium side,
an angle between the central axis of said first quadrate zone reposition pull screw hole and the long axis of the body is 88.6°, and the direction of the angle is downward, an angle between the central axis of said first quadrate zone reposition pull screw hole and the coronary axis of the body is 32.0°, and the direction of the angle is inward, an angle between the central axis of said first quadrate zone reposition pull screw hole and the sagittal axis of the body is 58.0°, and the direction of the angle is forward, an angle between the central axis of said first quadrate zone reposition pull screw hole and the plane of the quadrate zone is 130°;
an angle between the central axis of said second quadrate zone reposition pull screw hole and the long axis of the body is 87.4°, and the direction of the angle is downward, an angle between the central axis of said second quadrate zone reposition pull screw hole and the coronary axis of the body is 24.38°, and the direction of the angle is inward, an angle between the central axis of said second quadrate zone reposition pull screw hole and the sagittal axis of the body is 65.8°, and the direction of the angle is forward, an angle between the central axis of said second quadrate zone reposition pull screw hole and the plane of the quadrate zone is 123°.

8. The anatomical locking guide steel plate according to claim 1, wherein, a first locking screw hole is provided in said head of the steel plate, and a third locking screw hole is provided in said tail of the steel plate;
an angle between the central axis of said first locking screw hole and the long axis of the body is 72.3°, and the direction of the angle is downward, an angle between the central axis of said first locking screw hole and the coronary axis of the body is 66.8°, and the direction of the angle is inward, an angle between the central axis of said first locking screw hole and the sagittal axis of the body is 27.9°, and the direction of the angle is forward,
an angle between the central axis of said third locking screw hole and the long axis of the body is 57.8°, and the direction of the angle is downward, an angle between the central axis of said third locking screw hole and the coronary axis of the body is 48.8°, and the direction of the angle is inward, an angle between the central axis of said third locking screw hole and the sagittal axis of the body is 58.0°, and the direction of the angle is forward.

9. The anatomical locking guide steel plate according to claim 8, wherein, a second locking screw hole is provided in said head of the steel plate, and said second locking screw hole is located under said first locking screw hole,
a fourth locking screw hole is provided in the tail of the steel plate, and said fourth locking screw hole is located under said third locking screw hole,
an angle between the central axis of said second locking screw hole and the long axis of the body is 88.0°, and the direction of the angle is downward, an angle between the central axis of said second locking screw hole and the coronary axis of the body is 48.3°, and the direction of the angle is inward, an angle between the central axis of said second locking screw hole and the sagittal axis of the body is 41.8°, and the direction of the angle is forward,
the central axis of said fourth locking screw hole is parallel to the central axis of said third locking screw hole.

10. An anatomical locking guide apparatus for a complicated acetabulum fracture, said apparatus includes the anatomical locking guide steel plate for the complicated acetabulum fracture according to claim 1 and a guide module,
a first connection hole is provided between the first quadrate zone reposition pull screw hole and the second quadrate zone reposition pull screw hole,
a groove is provided in the ventral side of said guide module for mating with said body of the steel plate, so as to combine with said steel plate,
a first guide screw hole, a second guide screw hole and a second connection hole are provided in said guide module, the first guide screw hole, the second guide screw hole and the second connection hole correspond to the first quadrate zone reposition pull screw hole, the second quadrate zone reposition pull screw hole and the first connection hole respectively,
an inner edge of said guide module is matched with the big incisura of the ischium.

11. The anatomical locking guide apparatus according to claim 10, wherein, said body of the steel plate has a width of 11 mm-17 mm.

12. The anatomical locking guide apparatus according to claim 10, wherein, the distance from said head of the steel plate to an apex of the acetabulum is 28 mm-32 mm.

13. The anatomical locking guide apparatus according to claim 10, wherein, the outer edge of said body of the steel plate is parallel to a bony edge of the acetabulum, and the distance to the bony edge of the acetabulum is 4 mm-6 mm.

14. The anatomical locking guide apparatus according to claim 10, wherein, an angle between the central axis of said acetabulum anterior column pull screw hole and the long axis of the body is 44.6°, and the direction of the angle is downward; an angle between the central axis of said acetabulum anterior column pull screw hole and the coronary axis of the body is 56.7°, and the direction of the angle is inward; an angle between the central axis of said acetabulum anterior column pull screw hole and the sagittal axis of the body is 64.1°, and the direction of the angle is forward.

15. The anatomical locking guide apparatus according to claim 10, wherein, an angle between the central axis of said Magic pull screw hole and the long axis of the body is 49.1°, and the direction of the angle is downward; an angle between the central axis of said Magic pull screw hole and the coronary axis of the body is 55.6°, and the direction of the angle is inward; an angle between the central axis of said Magic pull screw hole and the sagittal axis of the body is 59.9°, and the direction of the angle is backward.

16. The anatomical locking guide apparatus according to claim 10, wherein, said first quadrate zone reposition pull screw hole is located at the ilium side, and said second quadrate zone reposition pull screw hole is located at the ischium side,
an angle between the central axis of said first quadrate zone reposition pull screw hole and the long axis of the body is 88.6°, and the direction of the angle is downward, an angle between the central axis of said first quadrate zone reposition pull screw hole and the coronary axis of the body is 32.0°, and the direction of the angle is inward, an angle between the central axis of said first quadrate zone reposition pull screw hole and the sagittal axis of the body is 58.0°, and the direction of the angle is forward, an angle between the central axis of said first quadrate zone reposition pull screw hole and the plane of the quadrate zone is 130°;
an angle between the central axis of said second quadrate zone reposition pull screw hole and the long axis of the body is 87.4°, and the direction of the angle is downward, an angle between the central axis of said second quadrate zone reposition pull screw hole and the coronary axis of the body is 24.38°, and the direction of the angle is inward, an angle between the central axis of said second quadrate zone reposition pull screw hole and the sagittal axis of the body is 65.8°, and the direction of the angle is forward, an angle between the central axis of said second quadrate zone reposition pull screw hole and the plane of the quadrate zone is 123°.

17. The anatomical locking guide apparatus according to claim 10, wherein, a first locking screw hole is provided in said head of the steel plate, and a third locking screw hole is provided in said tail of the steel plate;
an angle between the central axis of said first locking screw hole and the long axis of the body is 72.3°, and the direction of the angle is downward, an angle between the central axis of said first locking screw hole and the coronary axis of the body is 66.8°, and the direction of the angle is inward, an angle between the central axis of said first locking screw hole and the sagittal axis of the body is 27.9°, and the direction of the angle is forward,
an angle between the central axis of said third locking screw hole and the long axis of the body is 57.8°, and the direction of the angle is downward, an angle between the central axis of said third locking screw hole and the coronary axis of the body is 48.8°, and the direction of the angle is inward, an angle between the central axis of said third locking screw hole and the sagittal axis of the body is 58.0°, and the direction of the angle is forward.

18. The anatomical locking guide apparatus according to claim 17, wherein, a second locking screw hole is provided in said head of the steel plate, and said second locking screw hole is located under said first locking screw hole,
a fourth locking screw hole is provided in the tail of the steel plate, and said fourth locking screw hole is located under said third locking screw hole,
an locking screw hole and the long axis of the locking screw hole and the long axis of the body is 88.0°, and the direction of the angle is downward, an angle between the central axis of said second locking screw hole and the coronary axis of the body is 48.3°, and the direction of the angle is inward, an angle between the central axis of said second locking screw hole and the sagittal axis of the body is 41.8°, and the direction of the angle is forward,
the central axis of said fourth locking screw hole is parallel to the central axis of said third locking screw hole.

19. A guide sleeve apparatus, wherein said guide sleeve apparatus is used with the anatomical locking guide steel plate for the complicated acetabulum fracture according to claim 1,
said guide sleeve apparatus includes an outer sleeve, a pointed cone puncturing element and an inner core,
the inner diameter of said outer sleeve is matched with the outer diameters of said pointed cone puncturing element and said inner core, threads are provided at a front end of said outer sleeve, and the threads are matched with the screw holes in said anatomical locking guide steel plate,
the head of said pointed cone puncturing element is a blunt nosed cone,
said inner core has a hollow structure, its inner diameter is matched with the outer diameter of a guide needle, the head of said inner core has a serrate structure with a slope.

20. The guide sleeve apparatus of claim 19, wherein said guide sleeve apparatus is used with the anatomical locking guide apparatus for the complicated acetabulum fracture,
said guide sleeve apparatus includes an outer sleeve, a pointed cone puncturing element and an inner core,
the inner diameter of said outer sleeve is matched with the outer diameters of said pointed cone puncturing element and said inner core, threads are provided at a front end of said outer sleeve, and the threads are matched with the screw holes in said anatomical locking guide steel plate and/or said guide module,
the head of said pointed cone puncturing element is a blunt nosed cone,
said inner core has a hollow structure, its inner diameter is matched with the outer diameter of a guide needle, the head of said inner core has a serrate structure with a slope.
